(19) United States
(12) Patent Application Publication
(10) Pub. No.: US 2011/0054411 A1
(43) Pub. Date: Mar. 3, 2011

(54) Patient-Contact Activated Needle Stick Safety Device

(75) Inventors: Philip Dowds, San Diego, CA (US); James M. Verespej, San Marcos, CA (US)

(73) Assignee: Safety Syringes, Inc.

(21) Appl. No.: 12/859,698

(22) Filed: Aug. 19, 2010

Related U.S. Application Data
(60) Provisional application No. 61/235,278, filed on Aug. 19, 2009.

(51) Int. Cl. A61M 5/32

(52) U.S. Cl. 604/198

(57) ABSTRACT

A device that is used in conjunction with a needle-based medication injection device (e.g. a prefilled syringe) that prevents needle stick injuries after the medication has been injected into a patient. The used needle is shielded by a cylindrical needle guard that surrounds and extends beyond the needle tip. In a preferred embodiment, before the needle is inserted into the patient, the needle guard projects forward to substantially hide visibility of the needle for safety and to reduce patient anxiety.

This application claims the benefit of provisional application Ser. No. 61/235,278 filed Aug. 19, 2009.

### GENERAL DESCRIPTION

The following describes a device that is used in conjunction with a needle-based medication injection device (e.g. a prefilled syringe) that prevents needle stick injuries after the medication has been injected into a patient. The used needle is shielded by a cylindrical needle guard that surrounds and extends beyond the needle tip. In a preferred embodiment, before the needle is inserted into the patient, the needle guard projects forward to substantially hide visibility of the needle to reduce patient anxiety. The elements of the design and how they function are described below.

### IN THE DRAWINGS

FIG. 1 illustrates the fully assembled device;

FIG. 2 illustrates the fully assembled device quarter section view;

FIG. 3 illustrates the exploded view of various components of the device;

FIG. 4 illustrates the needle guard body grooves showing a position of the main body protrusions during the steps of operating the device;

FIG. 5 illustrates rigid needle shield (RNS) removal;

FIG. 6 illustrates the device ready for medication dispensing;

FIG. 7 illustrates medication dispensing steps;

FIG. 8 is a cross section after an injection of medication;

FIG. 9 illustrates removal of the device and needle from the injection site;

FIG. 10 illustrates the final safety configuration;

FIG. 11 illustrates the main body window position prior to the removal of the RNS and RNS Removal Tool;

FIG. 12 provides an illustration after the RNS has been removed and the needle guard body has moved forward;

FIG. 13 is a detailed showing of the RNS removal tool improvement;

FIG. 14 illustrates the RNS removal tool radial CAM mechanism to engage RNS;

FIG. 15 illustrates the RNS removal tool axial CAM mechanism;

FIG. 16 shows the RNS removal tool retention tool, nominal orientation allowing syringe assembly; and

FIG. 17 shows the removal tool retention barb engaging proximal end of the RNS.

### DETAILED DESCRIPTION

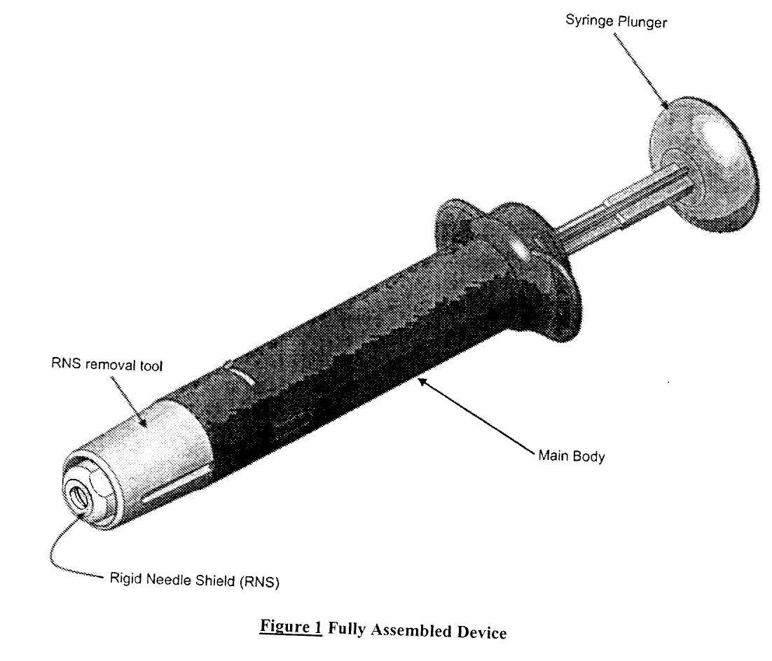

### FIG. 1 Fully Assembled Device

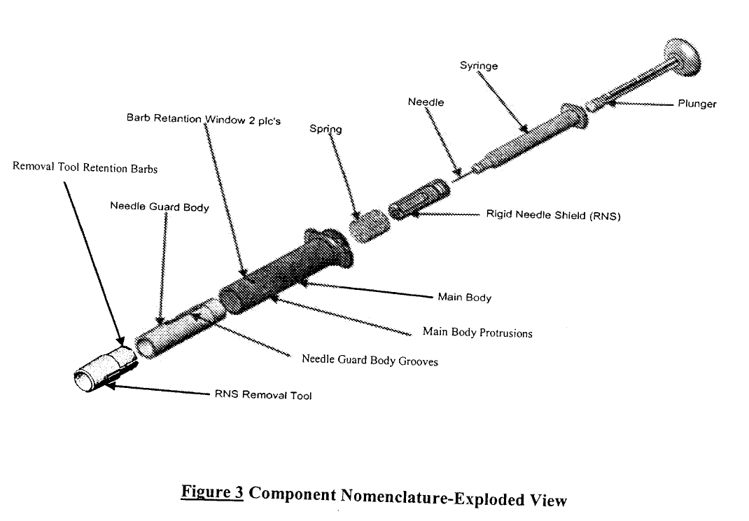

The fully assembled device is shown in FIG. 1. The assembly comprises of a Main Body with a Plunger emanating proximally from the proximal end. Inside the device is a prefilled syringe with an attached needle and a cover over the needle called a Rigid Needle Shield (RNS) that protects the needle and maintains a sterile barrier for the medication injection pathways. At the distal end of the assembly is the RNS Removal Tool, which is a removable covering over the RNS that facilitates its removal just before injecting medication into the patient. The plunger is attached to the syringe stopper. At the distal end of the assembly inside the Main Body is the Needle Guard Body, which is a tube that is concentric and interior to the Main Body (see FIGS. 3, 5, and 6).

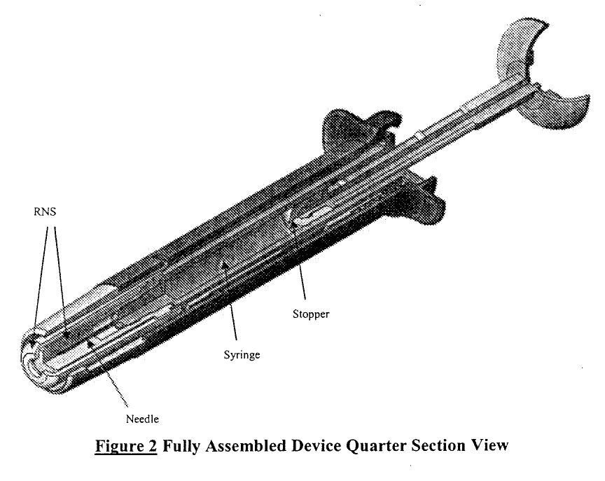

### FIG. 2 Fully Assembled Device Quarter Section View

The Needle Guard Body is axially slide-able with respect to the rest of the Main Body and is biased in a distal direction by a compression spring acting at its proximal end. Initially, the Needle Guard Body is held in a proximal position by the RNS Removal Tool. The RNS Removal Tool is held in this position against the force of the spring by Retention Barbs that project outwardly at the proximal end of the RNS Removal Tool and that mate with corresponding Retention Windows in the wall of the Main Body of the device.

Immediately before injecting medication into a patient, the RNS is removed by squeezing the RNS Removal Tool, which collapses along two slits that run along the side of the tool starting at its proximal end. The collapsed configuration of the tool allows the Retention Barbs at the proximal end to disengage from the corresponding Retention Windows in the Main Body of the device. Inwardly projecting RNS Barbs at the proximal inside surface of the RNS Removal Tool grasp the proximal edge of the RNS, which in combination with the compressive force transmitted by the collapsed RNS Removal Tool walls allows it to pull the RNS from the distal end of the syringe when the user pulls it in a distal direction.

### FIG. 3 Component Nomenclature-Exploded View

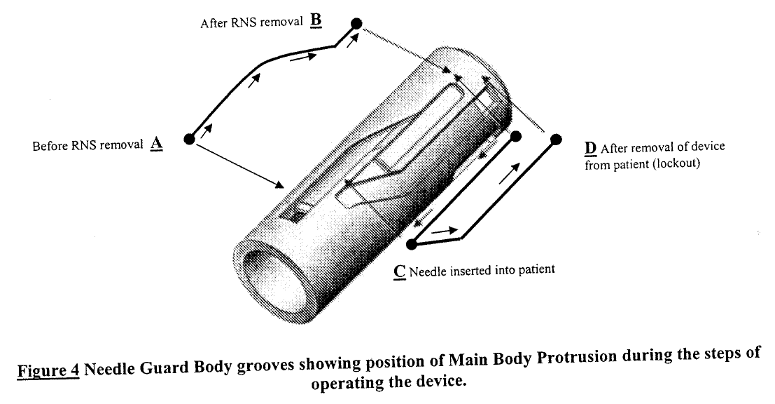

As the RNS Removal Tool is withdrawn from the end of the device, the Needle Guard Body slides forward to an intermediate stop point governed by the interference between one or more inwardly projecting Protrusions from the Main Body and corresponding grooves in the outer surface of the Needle Guard Body (see FIG. 4). The outer side of one Protrusion on the Main Body is shown in FIG. 3. They are positioned at the end of cut-out sections that provide flexibility to the Protrusions. The Protrusions interfere with the Needle Guard Body grooves by projecting into the groove space and controlling the movement of the Needle Guard Body against the distally directed force of the spring and the proximally directed reaction force from the patient's skin. Prior to removal of the RNS, the Protrusion is in position A of the Needle Guard Body groove as shown in FIG. 4. After the RNS is removed, the Needle Guard Body moves distally in response to the spring force, so that the Protrusion is at position B. As the needle is inserted into the patient, the patient's skin pushes the Needle Guard Body proximally against the spring force, such that the groove-protrusion interface moves from position B to position C. Toward the distal end of this groove section, the groove depth steps down to a deeper portion along an edge that is angled to the axis of the needle guard body. After injection is complete and the device is pulled away from the patient, the Needle Guard Body moves distally from position C such that the Protrusions encounter the stepped angled edge groove under the force of the spring. The angled stepped surface causes the Needle Guard Body to rotate with respect to the Main Body and to enter a final groove toward position D, wherein the Protrusion drops into a further deepening of the groove such that the protrusion is substantially captured. This engagement prevents relative motion of the Needle Guard Body with respect to the Main Body

At this point the Needle Guard Body has projected distally around the needle to the extent that it protects the caregiver and others from inadvertently being stuck by the needle tip. The Needle Guard Body is held in this needle-shielding position by interference of the Protrusions in the groove recess at position D so that the Needle Guard Body can not be pushed proximally with respect to the Main Body.

### FIG. 4 Needle Guard Body Grooves Showing Position of Main Body Protrusion During the Steps of Operating the Device.

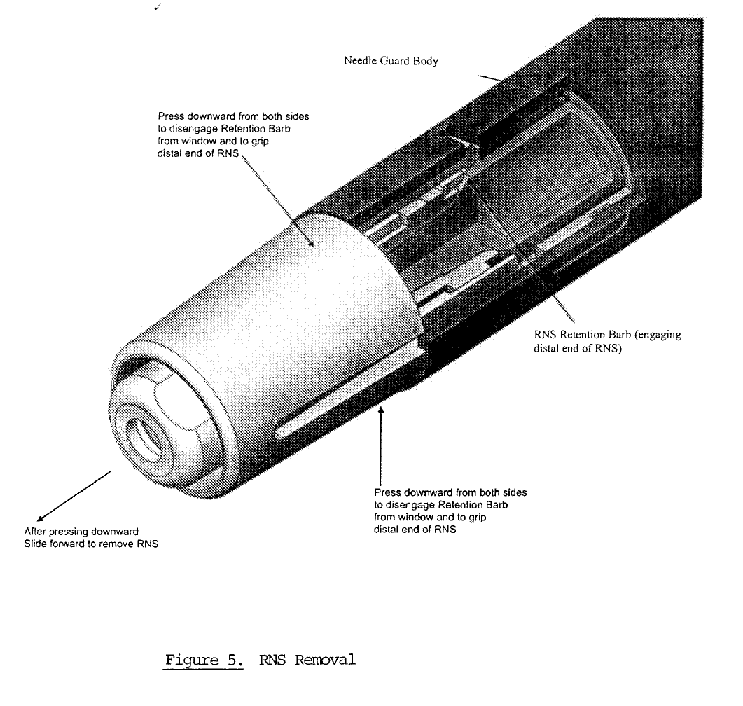

### FIG. 5 RNS Removal

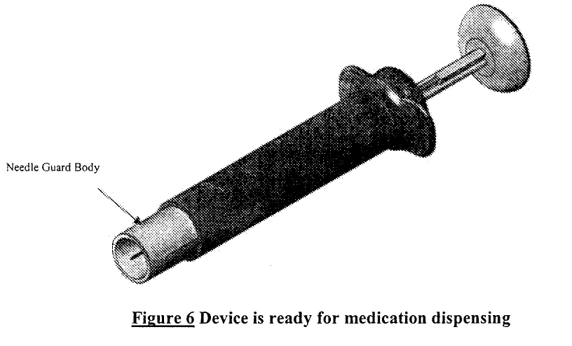

### FIG. 6 Device is Ready for Medication Dispensing

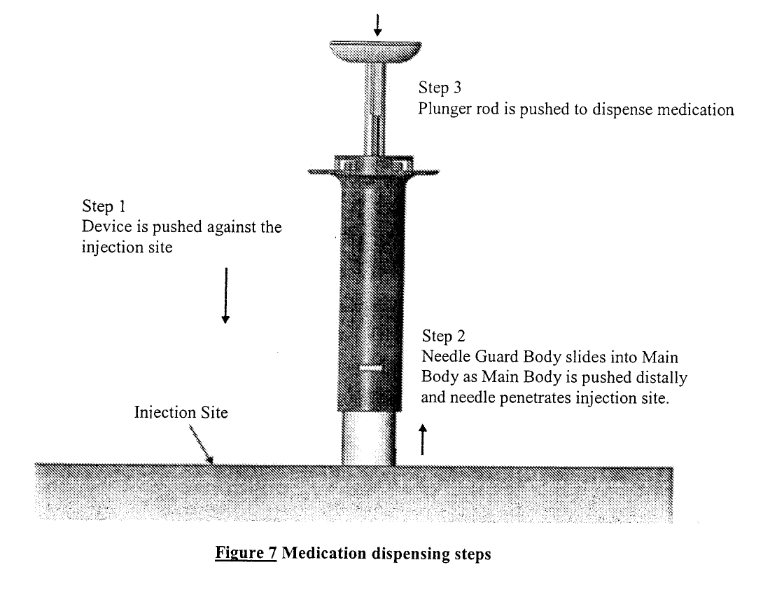

### FIG. 7 Medication Dispensing Steps

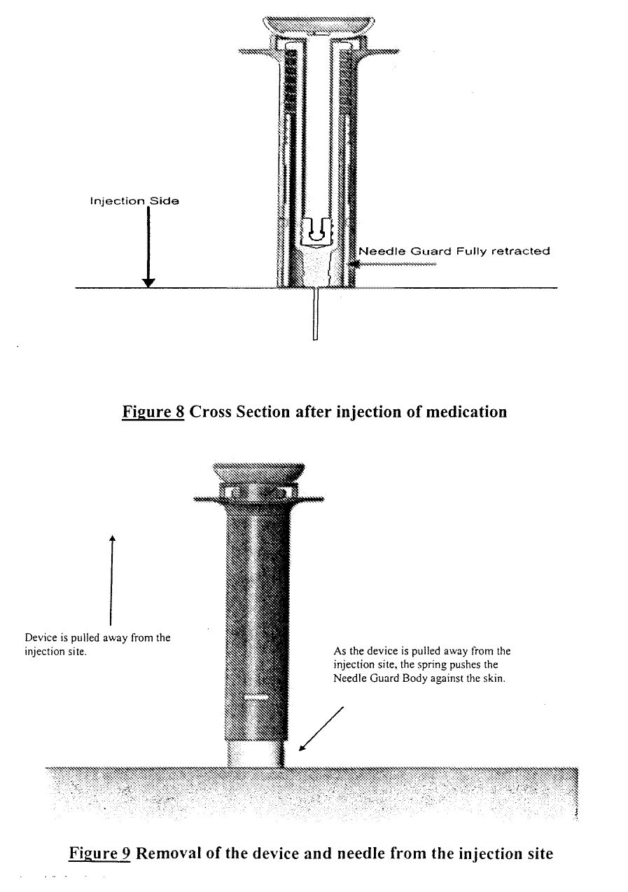

FIG. 8 Cross Section after Injection of Medication
FIG. 9 Removal of the Device and Needle from the Injection Site

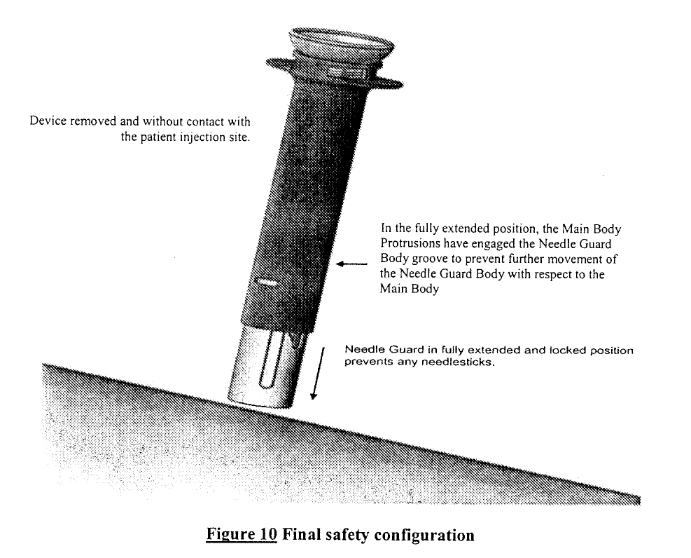

### FIG. 10 Final Safety Configuration

The sequence of steps to operate the device is described in FIGS. 5 through 10. The RNS Removal Tool is squeezed and pulled distally to remove the RNS as shown in FIG. 5. As a result of this, the Needle Guard Body moves distally to the position shown in FIG. 6. A composite of 3 steps to inject medication are shown in FIG. 7. In Step 1 the device is pushed against the patient's injection site. In Step 2, as the device is pushed against the injection site, the Needle Guard Body moves proximally allowing the needle to enter into the injection site. In Step 3, the Plunger Rod is pushed forward to dispel the medication into the injection site. FIG. 8 shows a cross-section of the device after the plunger has been fully depressed. In FIG. 9, the device is being withdrawn from the injection site while the spring pushes the Needle Guard Body distally. As the device is fully withdrawn from the injection site, the Needle Guard Body fully extends forward as shown in FIG. 10. At this point the Main Body Protrusions have entered position D in FIG. 4 and locked the Needle Guard Body from further motion.

To facilitate movement of the Main Body and its Protrusions with respect to the Grooves of the Needle Guard Body, one or both components can be made using a plastic resin with ample lubrication (e.g. high content of mold release). Alternatively, dissimilar plastic resins exhibiting a low mutual coefficient of friction can be used for the components.

It is to be understood that there exist alternative arrangements of components that would still fall within the scope of what is described and claimed within this application. For instance, the Needle Guard Body could be positioned on the outside of the main body with interior-facing grooves and outwardly facing protrusions on the main body.

### Light Protected Embodiment

An alternative embodiment for the safety device is presented for use with light-sensitive drugs that require only minimal exposure to light. In this embodiment, the Patient-Contact Activated Needle Stick Safety Device components are made of opaque materials (e.g. plastic resins with pigments, tinted glass, etc.) that effectively block light from reaching the drug in the medication delivery device. However, drug injection instructions normally require the caregiver to inspect the drug to check that it is not cloudy, etc. prior to giving the injection. To achieve this, the Main Body and Needle Guard Body of the device each have diametrically opposed windows that are positioned with respect to each other such that they are not aligned until the RNS Removal Tool and RNS have been removed. After removal, when the diametrically opposed windows on the two components align, they form a line of sight through the device, which enables the caregiver to inspect the drug volume. The RNS, RNS Removal Tool, and Plunger rod components would also be made of opaque materials to prevent light exposure at the ends of the device. A covering (not shown) over the proximal end of the syringe with a hole for the plunger rod could also be created to provide additional light protection.

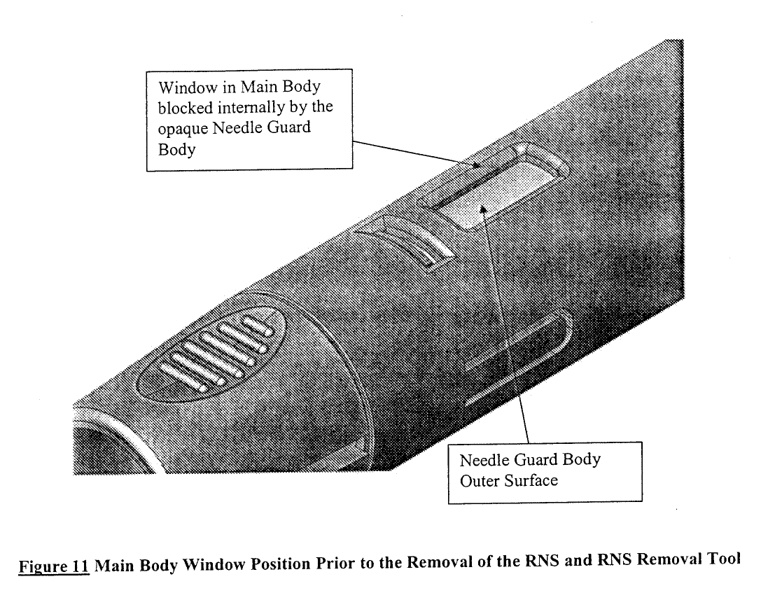

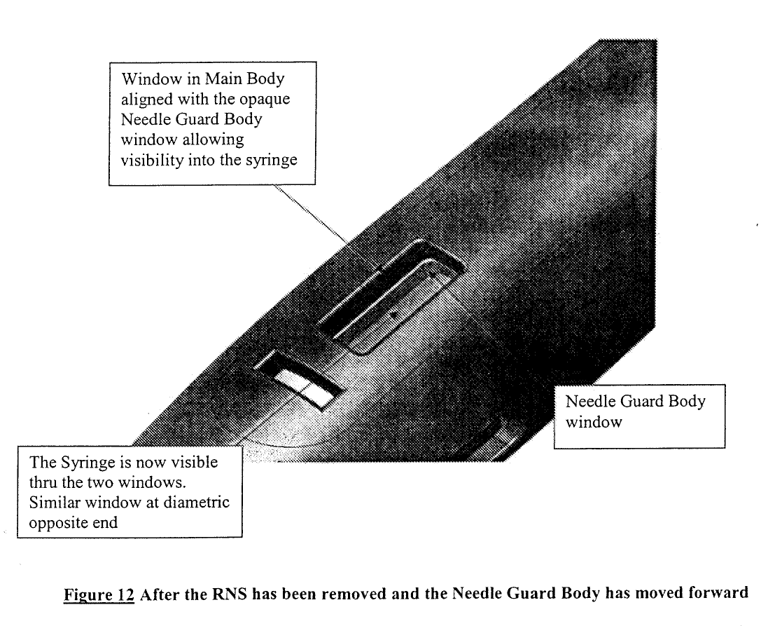

### FIG. 11 Main Body Window Position Prior to the Removal of the RNS and RNS Removal Tool

FIG. 12 After the RNS has been Removed and the Needle Guard Body has Moved Forward.

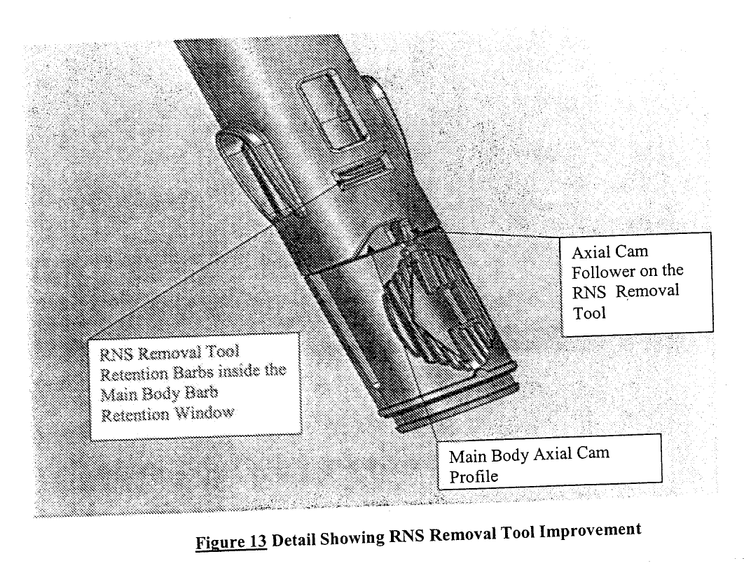

### FIG. 13 Detail Showing RNS Removal Tool Improvement

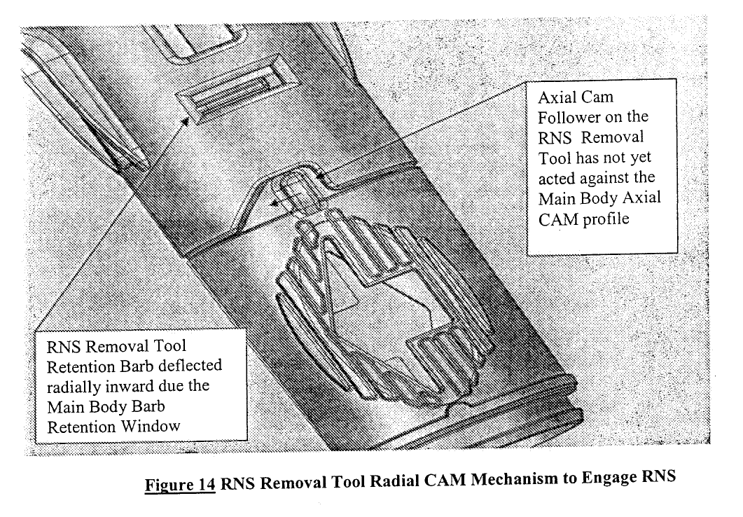

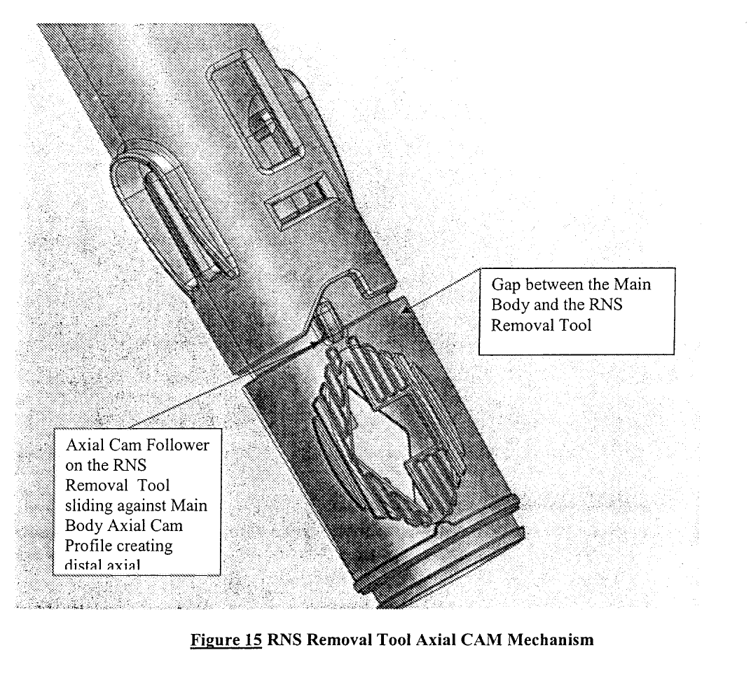

### FIG. 14 RNS Removal Tool Radial CAM Mechanism to Engage RNS

### FIG. 15 RNS Removal Tool Axial CAM Mechanism

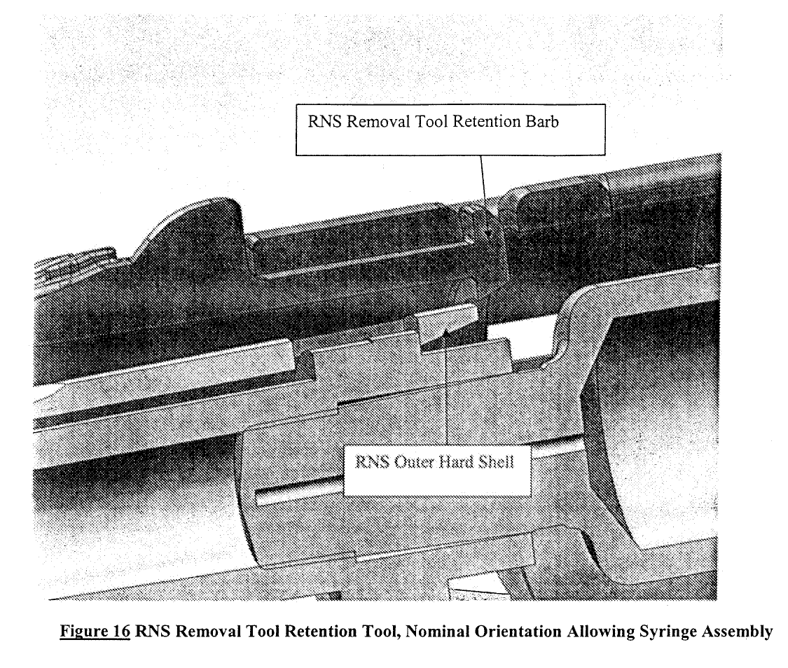

### FIG. 16 RNS Removal Tool Retention Tool, Nominal Orientation Allowing Syringe Assembly

The Rigid Needle Shield not only protects the needle from being bent or its tip from being damaged but it also forms one of the sterile barriers for the drug closure system. It must perform these functions before, during, and after sterilization and is therefore a complicated component that receives a tremendous amount of testing during drug development and approval process. Since it has potential contact with the drug inside the syringe, it becomes part of the specific drug closure system that receives regulatory approval and is therefore difficult to change after approval. They have become industry standard devices produced by specialized third party manufacturers. Nevertheless, they have limitations and deficiencies, namely that they can become difficult to remove from the syringe after sterilization and storage, often requiring greater than 20N of force to remove, which on a part so small (approximately 0.25 inches in diameter, 1 inch long) makes it difficult for healthcare workers to remove due to the small grasping area. Patients that perform self-administration, especially those with limited manual dexterity or strength (e.g. arthritic or multiple sclerosis patients) will find it extremely difficult to remove. Therefore, an added improvement of the present device is to facilitate the RNS removal. This is accomplished by the RNS Removal Tool, which in addition to presenting a bigger surface area with which the user can grab, it also features some CAM mechanisms to provide a mechanical leverage to removing the RNS. As shown in FIGS. 13, 14, and 16 the RNS Removal Tool Retention Barbs reside inside the Main Body Barb Retention Window before the RNS is removed. The lateral sides of this Barb and or the corresponding edges of the Main Body Barb Retention Window are angled such that as the RNS removal tool is rotated, the barbs push against the edge of the window and are deflected radially inward as shown in FIGS. 14 and 15.

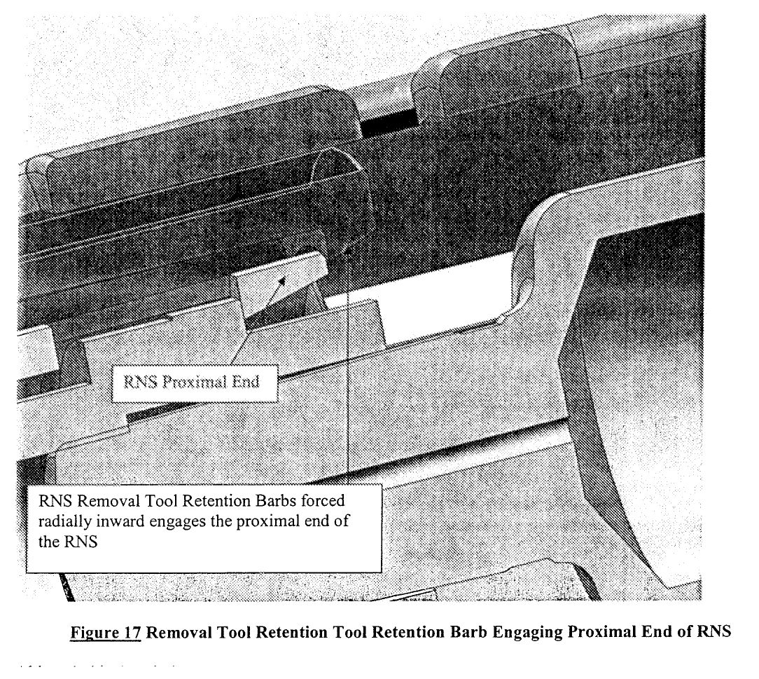

### FIG. 17 Removal Tool Retention Tool Retention Barb Engaging Proximal End of RNS

This mechanical advantage provides a strong radial squeeze so that the removal tool further engages the proximal edge of the RNS as shown in FIG. 17. This engagement cannot pre-exist sufficiently since the syringe and RNS must assemble into the device, from the proximal to distal end of device, with minimal resistance or disturbance to the RNS seal as shown in FIG. 16. After the proximal end of the RNS is engaged by the RNS Removal Tool, the Axial Cam Follower engages a sloped surface, the Main Body Axial Cam Profile as shown in FIGS. 15 and 17, which places a mechanically advantaged axial force on the RNS Removal Tool in a distal direction. Because the proximal end of the RNS is engaged, the RNS Removal Tool pushes the RNS off of the syringe and needle with much less effort on the part of the user than would normally be required.

Although this description has used a Rigid Needle Shield as an example, soft needle shields, which do not have a hard plastic outer shell, could equally be used in this application with minor changes to account for different geometry.

A further improvement to the device could be a distal end cap on the RNS Removal Tool and a proximal inwardly projecting lip that together would help contain the RNS after it had been removed from the syringe, preventing it from falling to the floor, etc.

The RNS Removal Tool can also have large cut-through arrows indicating the direction of rotation to the end user. It could also have large wings extending radially outward to provide greater rotational mechanical advantage for the end user.

For patients that have limited hand strength, holding the device against the skin while the needle is in the injection site, requires maintaining a force against the spring that pushes against the Needle Guard Body (position C in FIG. 4). A further improvement to the device would be to lessen this force by increasing the angle of the deeper groove section that starts proximally to point C in FIG. 4 and deflects the Main Body Protrusion over to the straight groove section that ends at D. In FIG. 4, this angle is shown at about 45 degrees. An angle of perhaps 60 degrees would place a greater axial component of force against the spring force at some reduction of the lateral force.

Of course, an angle of 90 degrees would hold the Needle Guard Body completely against the force of the spring if the Main Body Protrusion could stay down in the deeper section of the groove, but there would be no lateral deflection to get the Main Body Protrusion over to point D where the device locks out into the desired safety configuration.

Depending on the coefficient of friction between the Main Body Protrusion and the Needle Guard Body, the angle can be optimized to reduce the holding force for the patient, but still allow the Main Body Protrusion to lockout at point D of FIG. 4.

The glass syringe and rubber stopper have for years provided an ideal drug storage closure having unique properties of impermeability to oxygen, low extractables, biocompability, durability, etc. However they are both formed by processes that do not lend themselves to tight geometrical tolerances. For instance, the syringe flange is formed when a glass tube is heated to a soft state and the edges pressed over to form an edge. Typical tolerances for the inside length of a syringe or the length of a stopper are both +/−0.5 mm. The finger flange thickness has a similar tolerance. Furthermore, tight tolerances were not originally needed by these devices because they were not used mechanically with other devices. Existing passive anti-needle stick safety devices for prefilled syringes must mount to the syringe but not interfere excessively with the force required to move the plunger rod during injection nor prevent the full travel of the plunger rod. The safety mechanism necessarily must be triggered toward the end of administration of the drug (near the end of the plunger rod travel). However, since virtually all safety devices locate the syringe against the safety device at a point under the syringe finger flange, a stackup of worst-case tolerances can put the required plunger rod travel variance at +/−1.5 mm when considering just the tolerances of the inside length of the syringe, syringe flange thickness, and stopper length (syringe manufacturers reference the syringe length from the proximal end of the syringe, not the distal underside of the finger flange). To accommodate this 3 mm range of plunger rod position variance is very difficult for safety devices and it is a deliberate aim of the present invention to reduce and or eliminate any dependence of the safety device on the syringe and stopper tolerances. This is accomplished by having the safety device triggering mechanism independent of the syringe geometry. The present device is triggered when the Needle Guard Body is displaced proximally as the needle is inserted into the patient. The triggering point is broadly placed between point C in FIG. 4 and the angled step down feature proximal to point C. As long as the Needle Guard Body is pushed such that the Main Body Protrusion makes it to anywhere between point C and the angled step down, the device will lockout and this is almost completely independent of the syringe or stopper geometry.

The present safety device also makes the needle shielding completely contemporaneous with needle removal, reducing the possibility of needle stick injuries when, for instance, a patient suddenly jerks or flinches causing the needle to be come out of the patient before the plunger rod had fully traveled and activated the safety mechanism as would be the case with existing passive safety devices.

1. A needle stick safety device with a pre-filled syringe that prevents needle stick injuries after medication has been injected comprising
a main body for receiving a syringe or the like, a rigid needle shield on the end of the syringe, a removable covering over the shield for facilitating removal of shield just before injecting medication into a patient, a needle guard interior to the main body and slideable with respect to the main body and is biased in the distal direction by a compression spring.

2. A needle stick safety device as in claim 1 wherein the needle guard includes grooves for receiving a protrusion from the main body, and the main body including barb retention windows for engaging barbs on needle shield removable tool.

3. A needle stick safety device with a pre-syringe for preventing needle stick injuries after medication has been injected comprising
a rigid needle shield covering a needle of the syringe, a main body for receiving the syringe and rigid needle shield, a needle guard body within the main body for covering the needle of the syringe, and being movable to expose the needle within the main body as the needle guard body is pressed against the skin on a patient for injecting medication.

4. A devices as in claim 3 including a rigid shield removable tool for removing the rigid needle shield.
